(19) United States
(12) Patent Application Publication
(10) Pub. No.: US 2024/0293562 A1
(43) Pub. Date: Sep. 5, 2024

(54) METHODS FOR USING NEUROTROPHIN RECEPTOR BINDING CONJUGATE COMPOSITIONS IN THE TREATMENT OF A NEUROLOGICAL CONDITION

(71) Applicant: Manzanita Pharmaceuticals, Inc., Woodside, CA (US)

(72) Inventors: CONSTANCE A. MCKEE, Woodside, CA (US); STEPHEN B. KAHL, Woodside, CA (US); LOUIS EUGENE BURTON, San Mateo, CA (US); GORDON CRAIG HILL, New Market, MD (US)

(21) Appl. No.: 18/039,126

(22) PCT Filed: Nov. 30, 2021
(86) PCT No.: PCT/US2021/061167

§ 371 Date: May 26, 2023

Related U.S. Application Data
(60) Provisional application No. 63/119,471, filed on Nov. 30, 2020.
(60) Provisional application No. 63/161,419, filed on Mar. 15, 2021.

(51) Int. Cl. A61K 47/64; A61P 29/00

(52) U.S. Cl. CPC A61K 47/642; A61P 29/00

(57) ABSTRACT

Neurotrophin receptor binding conjugate compositions for treating a neurological condition (e.g., neuroinflammation caused by or associated with the neurological condition) are provided. Methods according to certain embodiments include administering to subject in need thereof an active agent conjugate that includes one or more active agent compounds for treating the neurological condition (e.g., neuroinflammation caused by or associated with the neurological condition) covalently bonded to a protein, peptide or peptidomimetic that binds selectively to a neurotrophin receptor. In certain embodiments. the active agent conjugate facilitates one or more of endocytosis, axonal transport and pharmacological activity in the neuronal cell body, such as when the active agent conjugate is administered to the subject intranasally. Kits for administering the active agent conjugate, such as intranasally, intracisternally or intravitreally are also described.

### CROSS-REFERENCE TO RELATED APPLICATIONS

Pursuant to 35 U.S.C. § 119(e), this application claims priority to the filing date of U.S. Provisional Patent Application No. 63/119,471, filed on Nov. 30, 2020 and U.S. Provisional Patent Application No. 63/161,419, filed on Mar. 15, 2021; the disclosure of which application is herein incorporated by reference.

### INTRODUCTION

Neuroinflammation is the inflammation of the nervous tissue, such as the brain and spinal cord. Neuroinflammation can be the brain's innate immune system that is triggered following an inflammatory challenge such as those posed by injury, infection, exposure to a toxin, neurodegenerative disease, or aging. This inflammation can be mediated by the production of reactive oxygen species, chemokines and cytokines. These mediators are produced by CNS glia including microglia and astrocytes as well as endothelial cells and peripherally derived immune cells. There are immune, physiological, biochemical, and psychological consequences of these neuroinflammatory responses. Aspects of neuroinflammation vary within the context of disease, injury, infection or stress.

Coronavirus disease-19 (COVID-19) is a worldwide health crisis. COVID-19 is caused by SARS-COV-2, a highly infectious pathogen that is genetically similar to SARS-CoV. Similar to other recent coronavirus outbreaks, including SARS and MERS, SARS-CoV-2 infected patients typically present with fever, dry cough, fatigue, and lower respiratory system dysfunction as well as pneumonia. It was initially thought that COVID-19 mainly affects the lungs, but it was soon reported that like SARS-COV and MERS-COV, some patients lose olfactory function. A rapidly accumulating set of clinical studies revealed atypical symptoms of COVID-19 that involve neurological signs, including headaches, anosmia, nausea, dysgeusia, damage to respiratory centers, and cerebral infarction. Up to an estimated 21.3% of COVID-19 patients exhibit CNS symptoms.

### SUMMARY

Neurotrophin receptor binding conjugate compositions for treating a neurological condition (e.g., neuroinflammation caused by or associated with the neurological condition) are provided. Methods according to certain embodiments include administering to subject in need thereof an active agent conjugate that includes one or more active agent compounds for treating the neurological condition (e.g., neuroinflammation caused by or associated with the neurological condition) covalently bonded to a protein, peptide or peptidomimetic that binds selectively to a neurotrophin receptor. In certain embodiments, the active agent conjugate facilitates one or more of endocytosis, axonal transport and pharmacological activity in the neuronal cell body, such as when the active agent conjugate is administered to the subject intranasally. Kits for administering the active agent conjugate, such as intranasally, intracisternally or intravitreally are also described.

Aspects of the present disclosure include neurotrophin receptor binding conjugate compositions for delivering an active agent compound for treating a neurological condition (e.g., neuroinflammation) to nerve cells. In some embodiments, the neurological condition comprises neuroinflammation caused by or associated with a brain injury, such as an acute brain injury or chronic brain injury. In some embodiments, the acute brain injury comprises a traumatic brain injury. In some embodiments, the brain injury comprises chronic traumatic encephalopathy. In other embodiments, the brain injury is caused by a pathogen (e.g., bacteria or virus). In certain embodiments, the brain injury is a hypoxia-induced brain injury.

In some embodiments, the neurological condition comprises neuroinflammation caused by or associated with brain cancer. In some instances, the brain cancer is a brain tumor, such as one selected from an astrocytoma, glioblastoma multiforme, meningioma, ependymoma, oligodendroglioma, cancer of the pituitary gland, craniopharyngioma, pineal region tumor and medulloblastoma. In certain instances, the brain cancer is caused by exposure to one or more toxins, such as a toxin released from burn pits.

In some embodiments, the neurological condition comprises multiple sclerosis. In some instances, the multiple sclerosis comprises relapse-remitting multiple sclerosis. In some instances, the multiple sclerosis comprises progressive multiple sclerosis. In certain instances, the multiple sclerosis comprises multiple sclerosis of viral etiology. In some embodiments, methods include treating neuroinflammation caused by or associated with multiple sclerosis.

In some embodiments, the neurological condition comprises neuroinflammation caused by or associated with Alzheimer's disease. In some embodiments, the neurological condition comprises neuroinflammation caused by or associated with Parkinson's disease. In some embodiments, the neurological condition comprises neuroinflammation caused by or associated with Huntington's disease. In some embodiments, the neurological condition comprises neuroinflammation caused by or associated with a prion disease. In some embodiments, the neurological condition comprises neuroinflammation caused by or associated with amyotrophic lateral sclerosis. In some embodiments, the neurological condition comprises neuroinflammation caused by or associated with frontotemporal dementia.

In some instances, the neuroinflammation comprises chronic pain. In some instances, the neuroinflammation is associated with or cause by one or more of headache, migraine, trigeminal neuralgia and herpes keratitis.

In some embodiments, the neurological condition is neuroinflammation caused by an infectious disease. In some instances, the infectious disease is a viral infection. In certain instances, the viral infection comprises infection by one or more of Herpes Zoster Virus (VZV) infection, Herpes Simplex Virus (HSV-1 or HSV-2), SARS-COV-2 virus or a variant thereof, Epstein-Barr virus, Hepatitis B virus, Hepatitis C virus, Human immunodeficiency virus type 1 (HIV-1), Human papilloma virus (HPV), Human T-cell lymphotropic virus type I (HTLV-1) and Kaposi sarcoma herpesvirus (KSHV). In certain cases, the neuroinflammation is associated with or caused by infection by SARS-COV-2 virus or a variant thereof.

In some embodiments, the subject is diagnosed as having neuroinflammation, such as by a health care professional. In some instances, methods of the present disclosure include treating neuroinflammation that includes encephalitis. In certain instances, the encephalitis is viral encephalitis, such as COVID-19 associated encephalitis. In other embodiments, the neuroinflammation includes inflammation of the optic nerve. In some embodiments, the the neuroinflammation includes acute disseminated encephalomyelitis, such as COVID-19 associated acute disseminated encephalomyelitis. In certain instances, the neuroinflammation includes inflammation of the vitreous retinal ganglion cells.

In embodiments, the active agent conjugates for treating neuroinflammation described herein include neurotrophin receptor conjugate compounds that bind a high affinity receptor. In some embodiments, the neurotrophin receptor conjugate compounds bind to a Trk receptor, such as for example TrkA receptors. In some embodiments, the neurotrophin receptor conjugate compounds bind to Trk receptors in a manner sufficient to facilitae endocytosis. In some embodiments, binding of the neurotrophin receptor conjugate compounds is sufficient to facilitate retrograde axonal transport of the anti-inflammatory agent of the neurotrophin receptor conjugate compound. In some embodiments, binding of the neurotrophin receptor conjugate compounds is sufficient to facilitate anterograde axonal transport of the anti-inflammatory agent of the neurotrophin receptor conjugate compound. In some embodiments, binding of the neurotrophin receptor conjugate compounds is sufficient to facilitate intracellular uptake of the anti-inflammatory agent, such as, for example, uptake of the anti-inflammatory agent into the neuronal cell body. In some embodiments, binding of the neurotrophin receptor conjugate compound is sufficient to facilitate intraneuronal transport of the anti-inflammatory agent. In some embodiments, binding of the neurotrophin receptor conjugate compound is sufficient to facilitate semi-intraneuronal transport of the anti-inflammatory agent. In some embodiment, semi-intraneuronal transport of anti-inflammatory agent includes transport along the myelin nerve sheath. In some embodiments, uptake into the neuronal cell body is sufficient for the anti-inflammatory agent to act intracellularly, such as, for example, where the anti-inflammatory agent exhibits anti-inflammatory pharmacological activity. In certain embodiments, methods described herein include administering a neurotrophin receptor conjugate compound for treating neuroinflammation where the administered conjugate compound exhibits one or more of: 1) binding to a receptor (e.g., a TrkA receptor): 2) retrograde or anterograde axonal transport of the anti-inflammatory agent of the neurotrophin receptor conjugate compound: and 3) uptake of the anti-inflammatory agent into the neuronal cell body or semi-intraneuronal transport of the anti-inflammatory agent such as transport along the myelin nerve sheath.

In embodiments, the protein, peptide or peptidomimetic component of conjugates in the subject compositions may be varied and may include, but is not limited to, nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF), and derivatives or fragments thereof. In some embodiments, the protein, peptide or 25 peptidomimetic component of the subject conjugates are mammalian derived proteins, such as mammalian nerve growth factor, mammalian brain-derived neurotrophic factor or mammalian neurotrophic factor. In certain instances, the protein, peptide or peptidomimetic component of the subject conjugates are not bacterial derived proteins, such as E. coli-derived nerve growth factor, E. coli-derived brain-derived neurotrophic factor or E. coli-derived neurotrophic factor. In certain embodiments, conjugates of interest include a combination of two or more protein, peptide or peptidomimetic components, such as two or more selected from nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF), and derivatives or fragments thereof. In some embodiments, the protein, peptide or peptidomimetic is a neurotrophic factor derived from a non-human animal. In other embodiments, the protein, peptide or peptidomimetic is a recombinant human neurotrophic factor. In still other embodiments, the protein, peptide or peptidomimetic is a subunit of a protein, such as a β-subunit of either NGF, rhNGF or rhBDNF, or a chimeric combination of one or more NTs. In some instances, the protein, peptide or peptidomimetic is a native neurotrophic factor (e.g., native NGF, native BDNF). In certain instances, the native neurotrophic factor does not include any additional peptide conjugates that have been functionalized for conjugating to the anti-inflammatory agent. As described in greater detail below, neurotrophin binding conjugate compounds of the present disclosure according to certain embodiments include a native protein such as a native nerve growth factor (NGF), a native brain-derived neurotrophic factor (BDNF) or a native neurotrophic factor that is conjugated to the anti-inflammatory agent compound through one or more amino acid residues (e.g., lysine residues) present in the native nerve growth factor (NGF), native brain-derived neurotrophic factor (BDNF) or native neurotrophic factor. In other words, according to these embodiments, the neurotrophin binding component does not include any non-natural or additional peptide sequences. In some instances, the protein, peptide or peptidomimetic component of subject conjugates does not include any additional C-terminal peptide sequences, such as for conjugating to the anti-inflammatory agent. In other instances, the protein, peptide or peptidomimetic component of subject conjugates does not include any additional N-terminal peptide sequences, such as for conjugating to the anti-inflammatory agent.

In certain embodiments, the protein, peptide or peptidomimetic is a neurotrophic factor that binds to tropomyosin kinase A (TrkA). In certain embodiments, the protein, peptide or peptidomimetic is a neurotrophic factor that exhibits retrograde axonal transport of the anti-inflammatory agent conjugated to the protein, peptide or peptidomimetic. In certain embodiments, the protein, peptide or peptidomimetic is a neurotrophic factor that exhibits intraneuronal uptake, such as into the neuronal cell body, axon or dendrite of the anti-inflammatory agent conjugated to the protein, peptide or peptidomimetic.

In embodiments, active agent conjugates in the subject compositions include an anti-inflammatory agent that treats neuroinflammation. In some embodiments, the anti-inflammatory agent is a glucocorticoid. In certain instances, the glucocortoid is fluocinolone acetonide. In other embodiments, active agent conjugates in the subject compositions include an antimicrobial compound. In certain instances, the antimicrobial compound is an antiviral compound. In some embodiments, the antiviral compound comprises plitidepsin. In some embodiments, active agent conjugates in the subject compositions include an anti-cancer compound. In certain instances, the active agent compound comprises bexarotene or a pharmaceutically acceptable salt thereof. In certain embodiments, the active agent is a compound configured to block receptor binding of neuropilin-1 (NRP1). In one example, the active agent is an antibody configured to block receptor binding of NRP1. In another example, the active agent is a small molecule compound configured to block receptor binding of NRP1. Active agent compounds of interest also include antibodies, such as monoclonal antibodies. In some instances, the active agent conjugate includes an antibody having activity (e.g, neutralizing activity) against one or more viruses. In certain instances, the active agent conjugate includes an antibody having activity (e.g., neutralizing activity) against the SARS-COV-2 virus or a variant thereof.

In some embodiments, the active agent component (e.g., anti-inflammatory agent such as fluocinolone acetonide) is associated with the protein, peptide or peptidomimetic component through a direct bond. In certain embodiments, the active agent component is covalently bonded directly to the protein, peptide or peptidomimetic component. In some embodiments, the active agent component is conjugated to the protein, peptide or peptidomimetic component through a bond to an internal amino acid residue of the protein, peptide or peptidomimetic. In other instances, the active agent component may be bonded to an intrachain amino acid residue along the surface of the protein, peptide or peptidomimetic. In still other instances, the active agent component may be bonded to a residue found at a binding site of the protein, peptide or peptidomimetic. In yet other instances, the active agent residue may be bonded to the protein, peptide or peptidomimetic component through an N-terminal amino acid or a C-terminal amino acid of the protein, peptide or peptidomimetic. In some embodiments, the active agent component is bonded to a mutated amino acid (e.g., amino acid substitution, non-natural amino acid, etc.) of the native protein, peptide or peptidomimetic. In other embodiments, the active agent component is bonded to a reactive amino acid of the native protein, peptide or peptidomimetic. For example, the linker may be bonded to a lysine residue of the protein, peptide or peptidomimetic. The active agent component may be bonded to a carbon or non-carbon atom of the protein, peptide or peptidomimetic. In one example, the active agent component is bonded to a carbon atom of the protein, peptide or peptidomimetic. In another example, the active agent component is bonded to a non-carbon atom of the protein, peptide or peptidomimetic, such as a nitrogen or sulfur atom of the protein, peptide or peptidomimetic.

In some embodiments, the active agent component and the protein, peptide or peptidomimetic component are associated together through one or more linkers. When present, the linker may be a cleavable linker or a non-cleavable linker. In some instances, the linker is a cleavable linker, such as an acid-cleavable linker, a base-cleavable linker, a photo-cleavable linker or an enzyme-cleavable (e.g., peptidase, esterase) linker. In certain instances, the linker includes a carbonate or carbmate moiety. In other instances, the linker is a non-cleavable linker. The linker may be a zero-length crosslinker, homobifunctional linker, heterobifunctional linker or a trifunctional crosslinker. In some embodiments, the linker may be used to conjugate the active agent component to the protein, peptide or peptidomimetic component through an internal amino acid residue of the protein, peptide or peptidomimetic. In other instances, the linker may be bonded to an intrachain amino acid residue along the surface of the protein, peptide or peptidomimetic. In still other instances, the linker may be bonded to a residue found in at a binding site of the protein, peptide or peptidomimetic. In yet other instances, the linker may be used to conjugate the active agent component to the protein, peptide or peptidomimetic component through an N-terminal amino acid or a C-terminal amino acid of the protein, peptide or peptidomimetic. In some embodiments, the linker is bonded to a mutated amino acid of the native protein, peptide or peptidomimetic. In other embodiments, the linker is bonded to a reactive amino acid of the native protein, peptide or peptidomimetic. The type of linkage for bonding the linker to the protein, peptide or peptidomimetic may be an ether linkage, a disulfide linkage or an amino linkage. For example, the linker may be bonded to a lysine residue of the protein, peptide or peptidomimetic. The linker may be bonded to a carbon or non-carbon atom of the protein, peptide or peptidomimetic. In one example, the linker is bonded to a carbon atom of the protein, peptide or peptidomimetic. In another example, the linker is bonded to a non-carbon atom of the protein, peptide or peptidomimetic, such as a nitrogen or sulfur atom of the protein, peptide or peptidomimetic.

In some embodiments, the subject compositions are administered intranasally to the subject. In other instances, the subject compositions are administered topically, intraocularly to the subject. In other instances, the composition is administered by injection, intracisternally to the subject. In yet other instances, the composition is administered by injection, intrathecally to the subject. In still other instances, the composition is administered intravitreally, either via intraocular injection or by intravitreal implant to the subject. In yet other instances, the composition is administered topically or transdermally to the subject. In other instances, the composition is administered to the subject by injection, such as by subcutaneous injection, intramuscular injection, or intrathecal injection.

### SELECT DEFINITIONS

The following terms have the following meaning unless otherwise indicated. Any undefined terms have their art recognized meanings.

“Pharmaceutical composition” refers to at least one compound and can further comprise a pharmaceutically acceptable carrier, with which the compound is administered to a patient.

“Pharmaceutically acceptable salt” refers to a salt of a compound, which possesses the desired pharmacological activity of the compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxy benzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like: or (2) salts formed when an acidic proton present in the compound is replaced by a metal ion, e.g., an alkali metal ion, an alkaline earth ion, or an aluminum ion: or coordinates with an organic base such as ethanolamine, diethanolamine, triethanolamine, N-methylglucamine and the like.

The term “solvate” as used herein refers to a complex or aggregate formed by one or more molecules of a solute, e.g. a conjugate compound or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

“Pharmaceutically acceptable carrier” refers to a diluent, adjuvant, excipient or vehicle with, or in which a compound is administered.

“Therapeutically effective amount” means the amount of a compound (e.g., conjugate) that, when administered to a patient, is sufficient to effect such treatment. The “therapeutically effective amount” will vary depending on the compound, the condition and its severity, and the age, weight, etc., of the patient.

### BRIEF DESCRIPTION OF THE FIGURES

The invention may be best understood from the following detailed description when read in conjunction with the accompanying drawings. Included in the drawings are the following figure:

FIG. 1 depicts mass spectrogram of FA-rhNGF according to certain embodiments.

FIG. 2 depicts sustains neuronal survival in superior cervical ganglia with FA-rhNGF according to certain embodiments.

FIGS. 3A and 3B depict in vivo activity of FA-rhNGF in a rat model of inflammatory pain according to certain embodiments. In CNA rats 10 days post-injury (DPI), FA-rhNGF injections reduce mechanical hyperalgesia, indicated by less pain-avoidance behavior in the MC test. No differences in the numbers of crossings were found when noxious probes were absent (0) mm). FA-rhNGF was injected IM into the hindpaw and calf (5X QD 6.6μg 7-11 DPI). On DPI 11 (last day of treatment), and DPI 16 (5 days after the end of treatment), sharp probes were elevated, reducing total number of crossings in all groups. FA-rhNGF-injected CNA rats crossed significantly more than vehicle-injected CNA rats on DPI 11 (P<0.05) with a trend for increased crossing on DPI 16.

FIG. 4 depicts suppression of in vitro SCI-induced hyperactivity of nociceptors by overnight incubation with FA-rhNGF according to certain embodiments.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a compound refers to one or more compounds. As such, the terms “a”, “an”, “one or more” and “at least one” can be used interchangeably. Similarly, the terms “comprising,” “including,” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Compounds as described herein can be purified by any of the means known in the art, including chromatographic means, such as high performance liquid chromatography (HPLC), preparative thin layer chromatography, flash column chromatography, and ion exchange (IEX) chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### METHODS FOR TREATING A NEUROLOGICAL CONDITION

As summarized above, aspects of the present disclosure include methods for treating a neurological condition, such as neuoroinflammation (e.g., encephalitis, encephalomyelitis). As described in greater detail below the subject active agent conjugates are administered to the subject in an amount sufficient to at least ameliorate the neurological condition. In certain embodiments the subject methods are sufficient to reduce or ameliorate at least one physical parameter of the neurological condition, which may not be discernible by the patient. In certain embodiments, the subject methods are sufficient to inhibit the neurological condition, either physically (e.g., stabilization of a discernible symptom), physiologically (e.g., stabilization of a physical parameter), or both. In certain embodiments, the subject methods are sufficient to delay the onset of the neurological condition. In some embodiments, the subject methods reduce or eliminate one or more symptoms of the neurological condition. In other embodiments the subject methods reduce the severity of the neurological condition, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 55% or more, such as by 60% or more, such as by 65% or more, such as by 70% or more, such as by 75% or more, such as by 80% or more, such as by 85% or more, such as by 90% or more, such as by 95% or more and including reducing the severity of the neurological condition by 97% or more. In other embodiments, the subject methods reduce the duration that the subject exhibits symptoms of the neurological condition, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 55% or more, such as by 60% or more, such as by 65% or more, such as by 70% or more, such as by 75% or more, such as by 80% or more, such as by 85% or more, such as by 90% or more, such as by 95% or more and including reducing the duration that the subject exhibits symptoms of the neurological condition by 97% or more. In certain instances, the subject methods reduce the risk of mortality caused by the neurological condition by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 55% or more, such as by 60% or more, such as by 65% or more, such as by 70% or more, such as by 75% or more, such as by 80% or more, such as by 85% or more, such as by 90% or more, such as by 95% or more and including reducing the subject methods reduce the risk of mortality caused by the neurological condition by 97% or more.

In some embodiments, the neurological condition is multiple sclerosis. In some instances, the multiple sclerosis comprises relapse-remitting multiple sclerosis. In some instances, the multiple sclerosis comprises progressive multiple sclerosis. In certain instances, the multiple sclerosis comprises multiple sclerosis of viral etiology. As described in greater detail below, treating multiple sclerosis according to embodiments of the disclosure include administering an active agent conjugate comprising an active agent covalently conjugated to a protein, peptide or peptidomimetic that binds selectively to a neurotrophin receptor. In certain embodiments, treating multiple sclerosis includes administering to the subject an active agent conjugate where the active agent comprises bexarotene or a pharmaceutically acceptable salt thereof. In certain instances, treating multiple sclerosis (e.g., relapse-remitting multiple sclerosis, progressive multiple sclerosis, etc.) includes administering the bexarotene active agent conjugate in a manner sufficient to facilitate retrograde or anterograde axonal transport of the bexarotene active agent conjugate. In some embodiments, binding of the bexarotene active agent conjugate is sufficient to facilitate one or more of: 1) transport of bexarotene along the myelin nerve sheath: 2) semi-intreaneuronal transport of bexarotene: and 3) intracellular uptake of bexarotene, such as, for example, uptake of bexarotene into the neuronal cell body.

In some embodiments, the neurological condition is brain cancer. In some embodiments, methods include treating a subject for neuroinflammation caused by or associated with the brain cancer. In some instances, the brain cancer is a brain tumor, such as one selected from an astrocytoma, glioblastoma multiforme, meningioma, ependymoma, oligodendroglioma, cancer of the pituitary gland, craniopharyngioma, pineal region tumor and medulloblastoma.

In some embodiments, the brain cancer is caused by exposure (chronic or acute) to one more toxins. In some instances, the one or more toxins is selected from the group consisting of acetaldehyde, acheson process, acid mists, aflatoxins, 4-aminobiphenyl, areca nut, aristolochic acid, arsenic and inorganic arsenic compounds, asbestos, auramine, azathioprine, benzene, benzidine and dyes metabolized to benzidine, benzo[a]pyrene, beryllium, betel quid, bis(chloromethyl)ether and chloromethyl methyl ether, busulfan, 1,3-Butadiene, cadmium, chlorambucil, chlornaphazine, chromium (VI), clonorchis sinensis, coal, coal gasification, coal-tar distillation, coal-tar pitch, coke production, cyclophosphamide, cyclosporine, 1,2-dichloropropane, diethylstilbestrol (DES), diesel engine exhaust, erionite, ethanol, ethylene oxide, etoposide, fluoro-edenite fibrous amphibole, formaldehyde, haematite, isopropyl alcohol manufacture using strong acids, leather dust, lindane, magenta, melphalan, methoxsalen (8-methoxypsoralen) plus ultraviolet A radiation (PUVA), methyl-CCNU, 4,4′-Methylenebis(chloroaniline) (MOCA), 2-naphthylamine, nickel, N′-Nitrosonornicotine (NNN) and 4-(N-Nitrosomethylamino)-1-(3-pyridyl)-1-butanone (NNK), 3,4,5,3′,4′-Pentachlorobiphenyl (PCB-126), 2,3,4,7,8-pentachlorodibenzofuran, pentachlorophenol, phenacetin, phosphorus-32, plutonium polychlorinated biphenyls (PCBs), dioxin, iodine-131, radium-224, radium-226, radium-228, radon-222, semustine (methyl-CCNU), 2,3,7,8-Tetrachlorodibenzo-para-dioxin (TCDD), ortho-Toluidine, treosulfan, trichloroethylene, vinyl chloride, welding fumes and wood dust. In certain instances, the brain cancer is caused by exposure to one or more toxins, such as a toxin released from burn pits.

In certain embodiments, the neurological condition is neuroinflammation. In embodiments, the term neuroinflammation is used herein in its conventional sense to refer to the inflammation of the nervous tissue, such as the brain and spinal cord. In certain embodiments, neuroinflammation includes inflammation of nervous tissue of the eyes, such as the vitreous retinal ganglion cells. In some embodiments, methods include treating neuroinflammation that includes neuroinflammation of the peripheral neurons. In some instances, neuroinflammation is triggered following an inflammatory challenge such as those posed by injury, infection, exposure to a toxin, neurodegenerative disease, or aging. This inflammation can be mediated by the production of reactive oxygen species, chemokines and cytokines. In certain embodiments, the neuroinflammation includes encephalitis. In some instances, the neuroinflammation is a pathogen-mediated encephalitis, such bacterial encephalitis or viral encephalitis. For example, methods of interest may include treating COVID-19 associated encephalitis. In other embodiments, the neuroinflammation includes inflammation of the optic nerve. In certain instances, the neuroinflammation includes acute disseminated encephalomyelitis. In some instances, the neuroinflammation is a pathogen-mediated encephalomyelitis, such bacterial encephalomyelitis or viral encephalomyelitis. For example, methods of interest may include treating COVID-19 associated encephalomyelitis. In certain embodiments, the neuroinflammation includes inflammation of vitreous retinal ganglion cells. In some embodiments, the neurological condition comprises neuroinflammation caused by or associated with Alzheimer's disease. In some embodiments, the neurological condition comprises neuroinflammation caused by or associated with Parkinson's disease. In some embodiments, the neurological condition comprises neuroinflammation caused by or associated with Huntington's disease. In some embodiments, the neurological condition comprises neuroinflammation caused by or associated with a prion disease. In some embodiments, the neurological condition comprises neuroinflammation caused by or associated with amyotrophic lateral sclerosis. In some embodiments, the neurological condition comprises neuroinflammation caused by or associated with frontotemporal dementia.

In some instances, the neuroinflammation comprises chronic pain. In some instances, the neuroinflammation is associated with or cause by one or more of headache, migraine, trigeminal neuralgia and herpes keratitis. In some embodiments, the neurological condition is neuroinflammation caused by an infectious disease. In some instances, the infectious disease is a viral infection. In certain instances, the viral infection comprises infection by one or more of Herpes Zoster Virus (VZV) infection, Herpes Simplex Virus (HSV-1 or HSV-2), SARS-COV-2 virus or a variant thereof, Epstein-Barr virus, Hepatitis B virus, Hepatitis C virus, Human immunodeficiency virus type 1 (HIV-1), Human papilloma virus (HPV), Human T-cell lymphotropic virus type I (HTLV-1) and Kaposi sarcoma herpesvirus (KSHV).

In certain cases, the neuroinflammation is associated with or caused by infection by SARS-COV-2 virus or a variant thereof. In certain cases, methods include treating a subject with COVID-19 caused by SARS-COV-2 coronavirus infection where the virus has one or more mutations. In some cases, the subject is infected with SARS-COV-2 coronavirus having one or more mutations selected from K417N, L452R, E484K, N501K, N501Y, D614G and P681H. In some cases, methods include treating a subject with COVID-19 that is caused by infection by one or more of SARS-COV-2 coronavirus of lineage B.1.1.207 (having a P681H mutation), SARS-COV-2 coronavirus of lineage B.1.1.7 (having a N501Y mutation), SARS-CoV-2 coronavirus Cluster 5, SARS-COV-2 coronavirus variant 501. V2 (having N501Y, K417N and E484K mutations), SARS-COV-2 coronavirus of lineage P.1 (having N501Y and E484K mutations) and SARS-COV-2 coronavirus of lineage B.1.429 (having L452R mutation).

In practicing the subject methods, treating neuroinflammation as described herein, refers, in certain embodiments, to ameliorating the neuroinflammation (i.e., arresting or reducing the development of the neuroinflammation). In certain embodiments “treating” or “treatment” refers to ameliorating at least one physical parameter, which may not be discernible by the patient. In certain embodiments, “treating” or “treatment” refers to inhibiting the condition, either physically (e.g., stabilization of a discernible symptom), physiologically (e.g., stabilization of a physical parameter), or both. In certain embodiments, “treating” or “treatment” refers to delaying the onset of the condition. In some embodiments, the subject methods reduce or eliminate one or more symptoms of neuroinflammation. In other embodiments the subject methods reduce the duration of neuroinflammation, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 55% or more, such as by 60% or more, such as by 65% or more, such as by 70% or more, such as by 75% or more, such as by 80% or more, such as by 85% or more, such as by 90% or more, such as by 95% or more and including reducing the duration of the inflammation by 97% or more. In other embodiments, the subject methods reduce the duration that the subject exhibits symptoms of neuroinflammation, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 55% or more, such as by 60% or more, such as by 65% or more, such as by 70% or more, such as by 75% or more, such as by 80% or more, such as by 85% or more, such as by 90% or more, such as by 95% or more and including reducing the duration that the subject exhibits symptoms of neuroinflammation by 97% or more.

In some embodiments, the neurological condition comprises neuroinflammation caused by or associated with a brain injury, such as an acute brain injury or chronic brain injury. In some embodiments, the acute brain injury comprises a traumatic brain injury. In some embodiments, the brain injury comprises chronic traumatic encephalopathy. In other embodiments, the brain injury is caused by a pathogen, such as bacteria, a virus or other microbe (e.g., an amoeba). In certain embodiments, the brain injury is a hypoxia-induced brain injury.

As summarized above, methods of the present disclosure include administering to a subject diagnosed as having, or exhibiting at least one symptom of neuroinflammation an active agent conjugate that includes one or more active agent compounds that treats neuroinflammation covalently bonded to a protein, peptide or peptidomimetic that binds selectively to a neurotrophin receptor. In certain embodiments, compounds of interest include conjugates such as those depicted by formula: X-L-B, where X is an active agent such as a small molecule or biopolymer (e.g., antibody, enzyme) anti-inflammatory; B is a protein, peptide or pepetidomimetic that binds selectively to a neurotrophin receptor; and L is an optional linker.

Conjugate compounds of interest include a protein, peptide or peptidomimetic that facilitates one or more of binding. endocytosis and transport (e.g., retrograde axonal transport to neuronal cell body) by a neurotrophin receptor. In some embodiments, the protein, peptide or peptidomimetic is a component that facilitates binding of the conjugate compound to a neurotrophin receptor. In other embodiments, the protein, peptide or peptidomimetic is a component that facilitates binding and endocytosis of the conjugate compound by a neurotrophin receptor. In vet other embodiments, the protein, peptide or peptidomimetic is a component that facilitates binding. endocytosis and transport (e.g., retrograde axonal transport, anterograde axonal transport) of the conjugate compound by a neurotrophin receptor. Protein, peptide or peptidomimetics of interest include, but are not limited to, nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF): derivatives, analogs, and fragments thereof such as recombinant molecules of NGF, BDNF, GDNF, CNTF, as well as synthetic peptides that bind to nerve cell surface receptors and have agonist or antagonist activities of growth factors. The protein, peptide or peptidomimetic may be derived from a non-human animal or may be recombinant human form expressed and produced either in bacterial systems e.g., E. coli cells or in mammalian cell systems, such as for example CHO (Chinese Hamster Ovary) cells. In some embodiments, the protein, peptide or peptidomimetic component of the subject conjugates are mammalian derived proteins, such as mammalian nerve growth factor, mammalian brain-derived neurotrophic factor or mammalian neurotrophic factor. In certain instances, the protein, peptide or peptidomimetic component of the subject conjugates are not bacterially derived proteins, such as E. coli-derived nerve growth factor, E. coli-derived brain-derived neurotrophic factor or E. coli-derived neurotrophic factor. In certain embodiments, the protein, peptide or peptidomimetic is a subunit of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF), such as a B-subunit of NGF, rhNGF or rhBDNF, or a chimeric combination of one or more NTs. In some instances, the protein, peptide or peptidomimetic is a native neurotrophic factor (e.g., native NGF, native BDNF). In certain instances, the native neurotrophic factor does not include any additional peptide conjugates that have been functionalized for conjugating to the anti-inflammatory agent. As described in greater detail below; neurotrophin binding conjugate compounds of the present disclosure according to certain embodiments include a native protein such as a native nerve growth factor (NGF), a native brain-derived neurotrophic factor (BDNF) or a native neurotrophic factor that is conjugated to the active agent compound through one or more amino acid residues (e.g., lysine residues) present in the native nerve growth factor (NGF), native brain-derived neurotrophic factor (BDNF) or native neurotrophic factor. In other words, according to these embodiments, the neurotrophin binding component does not include any non-natural or additional peptide sequences. In some instances, the protein, peptide or peptidomimetic component of subject conjugates does not include any additional C-terminal peptide sequences, such as for conjugating to the anti-inflammatory agent. In other instances, the protein, peptide or peptidomimetic component of subject conjugates does not include any additional N-terminal peptide sequences, such as for conjugating to the anti-inflammatory agent.

In certain embodiments, the protein, peptide or peptidomimetic is a neurotrophic factor that binds tropomyosin kinase A (TrkA). In certain embodiments, the protein, peptide or peptidomimetic is a neurotrophic factor that exhibits retrograde axonal transport of an anti-inflammatory agent conjugated to the protein, peptide or peptidomimetic. In certain embodiments, the protein, peptide or peptidomimetic is a neurotrophic factor that exhibits uptake (e.g., into the neuronal cell body or cellular processes) of an anti-inflammatory agent conjugated to the protein, peptide or peptidomimetic.

Compositions of interest may include one or more types of protein, peptide or peptidomimetic conjugate compounds, such as two or more types, such as three or more types and including five or more types. For example, compositions may include one or more NGF-containing conjugates, BDNF-containing conjugates, NT-3-containing conjugates, NT-4/5-containing conjugates, NT-6-containing conjugates, NT-7-containing conjugates, GDNF-containing conjugates, CNTF-containing conjugates, or conjugates containing a chimeric construct of e.g. NGF-BDNF that preserves Trk binding activity after synthetic manipulation.

Conjugate compounds include one or more active agents for treating neuroinflammation. The active agent may be an anti-inflammatory compound, such as a glucocorticoid. In certain instances, the active agent is fluocinolone acetonide. In other instances, the active agent is an antimicrobial compound. In some embodiments, the active agent is an anti-inflammatory agent. In certain instances, the anti-inflammatory agent is a glucocorticoid, such as fluocinolone acetonide. In other embodiments, the active agent is an antimicrobial compound. Antimicrobial agents of interest may include but are not limited to antibacterials, antifungals, antivirals, antiparasitics as well as antimicrobial pesticides. For example, antimicrobials may include fluoroquinolones such as ciprofloxacin, norfloxacin, ofloxacin, enoxacin, perfloxacin, fleroxacin, enrofloxacin, marbofloxacin, sarafloxacin, orbifloxacin, danofloxacin: aminoglycosides such as streptomycin, netilmicin, kanamycin, neomycin, tobramycin, amikacin, sisomicin, ribostamycin, dibekacin, framycetin, gentamycin, penicillins and aminopenicillins such as penicillin, ampicillin, amoxicillin, nafcillin, oxacillin and ticarcillin, cephalosporins such as ceftriaxone, cephalexin, cefadroxil and ceftiofur, β-lactams such as clavulanic acid which may be used in conjunction with penicillins or aminopenicillins, macrolides such as clarythromycin and erythromycin and other antibiotics such as dactinomycin, clindamycin, naladixic acid, chloramphenicol, rifamopin, clofazimine, spectinomycin, polymyxin B, colistin, minocycline, vancomycin, hygromycin B or C, fusidic acid, trimethoprim and cefotaxim.

In some embodiments, antiviral compounds may include but are not limited to Ribavirin, Fenretinide, Favipiravir, Brincidofovir, ZMapp, TKM-100802, BCX4430, Interferons, Amiodarone, Atorvostatin, Irbesartan, Clomiphene, FX06, Zmab, Tamoxifen, Albendazole, AC-93253, Toremifene, T-705, and GS-5734 (remdesivir). In certain embodiments, the antiviral is plitidepsin.

In certain embodiments, the active agent is a compound configured to to block receptor binding of neuropilin-1 (NRP1). In some instances, the active agent is an antibody configured to block receptor binding of NRP1. In other instances, the active agent is a small molecule compound configured to block receptor binding of NRP1.

Active agent compounds in conjugates of interest also include antibodies, such as monoclonal antibodies. In some instances, the active agent conjugate includes a monoclonal antibody having activity (e.g, neutralizing activity) against one or more viruses, such as those described above. In certain instances, the active agent conjugate includes a monoclonal antibody having activity (e.g., neutralizing activity) against the SARS-COV-2 coronavirus or a variant thereof. In certain cases, the active agent conjugate includes a monoclonal antibody having activity against the SARS-COV-2 coronavirus that has one or more mutations. In some cases, the one or more mutations is selected from K417N, L452R, E484K, N501K, N501Y, D614G and P681H. In some cases, the active agent conjugate includes a monoclonal antibody having activity against the SARS-COV-2 coronavirus of lineage B.1.1.207 (having a P681H mutation), SARS-COV-2 coronavirus of lineage B.1.1.7 (having a N501Y mutation), SARS-COV-2 coronavirus Cluster 5, SARS-COV-2 coronavirus variant 501. V2 (having N501Y, K417N and E484K mutations), SARS-COV-2 coronavirus of lineage P.1 (having N501Y and E484K mutations) and SARS-COV-2 coronavirus of lineage B.1.429 (having L452R mutation).

In certain embodiments, the active agent conjugate includes a monoclonal antibody selected from bamlanivimab (LY-COV555), casirivimab, imdevimab and etesevimab or a combination thereof.

In some embodiments, the active agent component (e.g., anti-inflammatory agent) is associated with the protein, peptide or peptidomimetic component through a direct bond. In certain embodiments, the active agent component is covalently bonded directly to the protein, peptide or peptidomimetic component or biopolymer. In some embodiments, the active agent component is conjugated to the protein, peptide or peptidomimetic component through a bond to an internal amino acid residue of the protein, peptide or peptidomimetic. In other instances, the active agent component may be bonded to an intrachain amino acid residue along the surface of the protein, peptide or peptidomimetic. In still other instances, the active agent component may be bonded to a residue found at a binding site of the protein, peptide or peptidomimetic. In yet other instances, the active agent residue may be bonded to the protein, peptide or peptidomimetic component through an N-terminal amino acid or a C-terminal amino acid of the protein, peptide or peptidomimetic. In some embodiments, the active agent component is bonded to a mutated amino acid (e.g., substituted amino acid or non-natural amino acid) of the native protein, peptide or peptidomimetic. In other embodiments, the active agent component is bonded to a reactive amino acid of the native protein, peptide or peptidomimetic. For example, the active agent may be bonded to a lysine residue of the protein, peptide or peptidomimetic. The active agent component may be bonded to a carbon or non-carbon atom of the protein, peptide or peptidomimetic. In one example, the active agent component is bonded to a carbon atom of the protein, peptide or peptidomimetic. In another example, the active agent component is bonded to a non-carbon atom of the protein, peptide or peptidomimetic, such as a nitrogen or sulfur atom of the protein, peptide or peptidomimetic.

In certain embodiments, the active agent component is bonded to the protein, peptide or peptidomimetic component through a linker. The linker may be any convenient covalent linking protocol, such as a zero-length crosslinker, homobifunctional linker, heterobifunctional linker or a trifunctional crosslinker. The linker may include one or more functional groups, such as an amide, alkylamine, carbamate, carbonate, thiolether, alkyl, cycloalkyl or aryl moiety, as desired. In some embodiments, the linker includes a carbamate moiety. The linker may be used to conjugate the active agent component to the protein, peptide or peptidomimetic component through an N-terminal amino acid or a C-terminal amino acid of the protein, peptide or peptidomimetic. In some embodiments, the linker is bonded to a mutated amino acid of the native protein, peptide or peptidomimetic. The type of linkage for bonding the linker to the protein, peptide or peptidomimetic may be an ether linkage, a disulfide linkage or an amino linkage. For example, the linker may be bonded to a lysine residue of the protein, peptide or peptidomimetic. The linker may be bonded to a carbon or non-carbon atom of the protein, peptide or peptidomimetic. In one example, the linker is bonded to a carbon atom of the protein, peptide or peptidomimetic. In another example, the linker is bonded to a non-carbon atom of the protein, peptide or peptidomimetic, such as a nitrogen or sulfur atom of the protein, peptide or peptidomimetic.

In some embodiments, the linker is cleavable. The term “cleavable” is used herein in its conventional sense to refer to linkers that can be cleaved under predetermined conditions so as to break the bond between the active agent and the binding moiety. For example, the linker may be an acid-cleavable linker, a base-cleavable linker, a photo-cleavable linker or an enzyme-cleavable (e.g., peptidase, esterase) linker. Acid-cleavable linkers are cleaved by subjecting the conjugate compound to a pH of 7 or below, such as a pH of 6.5 or below, such as a pH of 6.0 or below, such as a pH of 5.5 or below, such as a pH of 5.0 or below, such as a pH of 4.5 of below, such as a pH of 4.0 or below, such as a pH of 3.5 or below, such as a pH of 3.0 or below, such as a pH of 2.5 or below; such a pH of 2.0 or below, such as a pH of 1.5 or below and including a pH of 1.0 or below. Base-cleavable linkers are cleaved by subjecting the conjugate compound to a pH of 7 or above, such as a pH of 7.5 or above, such as a pH of 8.0 or above, such as a pH of 8.5 or above, such as a pH of 9.0 or above, such as a pH of 9.5 of above, such as a pH of 10.0 or above, such as a pH of 10.5 or above, such as a pH of 11.0 or above, such as a pH of 11.5 or above, such a pH of 12.0 or above, such as a pH of 12.5 or above and including a pH of 13.0 or above.

In certain embodiments, the conjugate compounds in the subject compositions include an enzyme-cleavable linker. In some instances, the enzyme cleavable linker is cleaved by contacting the compound with a peptidase, such as trypsin or chymotrypsin. In other instances, the enzyme cleavable linker is cleaved by contacting the compound with an esterase. In some embodiments, linkers of interest include those described in U.S. Pat. Nos. 5,767,288: 5,563,250; 5,505,931 and 4,469,774, the disclosures of which are herein incorporated by reference.

In other embodiments, the conjugate compounds in the subject compositions include a non-cleavable linker. The term “non-cleavable” is used herein in its conventional sense to refer to a covalently bonded moiety that is stable under physiological conditions and does not release the active agent from the binding moiety (e.g., the anti-inflammatory agent remains covalently bonded to the protein, peptide or peptidomimetic that selectively binds the neurotrophin receptor). In other words, conjugate compounds administered according to the subject methods having non-cleavable linkers are not susceptible to cleavage by acid, base, light or treatment with an enzyme. In these embodiments, 90% or more of conjugate compounds in a composition subjected to treatment by acid, base, light or with an enzyme does not result in release of the active agent from the binding moiety (e.g., protein, peptide or peptidomimetic that selectively binds the nerve cells), such as 95% or more, such as 97% or more, such as 98% or more, such as 99% or more and including 99.9% or more of the conjugate compounds in a composition subjected to treatment by acid, base, light or with an enzyme does not result in release of the active agent from the binding moiety. Examples of suitable non-cleavable linkers may include, but are not limited to, maleimido-containing crosslinkers, such as: N-succinimidyl 4-(maleimidomethyl)cyclohexanecarboxylate (SMCC), N-succinimidyl-4-(N-maleimidomethyl)-cyclohexane-1-carboxy-(6-amidocaproate), κ-maleimidoundecanoic acid N-succinimidyl ester (KMUA), γ-maleimidobutyric acid N-succinimidyl ester (GMBS), ϵ-maleimidocaproic acid N-hydroxysuccinimide ester (EMCS), m-maleimidobenzoyl-N-hydroxysuccinimide ester (MBS), N-(α-maleimidoacetoxy)-succinimide ester [AMAS], succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI) or haloacetyl-containing crosslinkers, such as: N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido)propionate (SBAP).

Aspects of the present disclosure also include methods for treating a subject for neuroinflammation with one or more of the subject conjugate compounds. In describing methods of the present disclosure, the term “subject” is meant the person or organism to which the conjugate compound is administered. As such, subjects of the present disclosure may include but are not limited to mammals, e.g., humans and other primates, such as chimpanzees and other apes and monkey species, dogs, rabbits, cats and other domesticated pets: and the like, where in certain embodiments the subject are humans. The term subject is also meant to include a person or organism of any age, weight or other physical characteristic, where the subjects may be an adult, a child, an infant, or a new born.

Methods according to certain embodiments include administering to a subject diagnosed as having neuroinflammation or exhibiting at least one symptom of neuroinflammation a conjugate compound having one or more active agent compounds (e.g., an anti-inflammatory agent) covalently bonded to a protein, peptide or peptidomimetic that binds selectively to a neurotrophin receptor of a nerve cell in the subject. In some embodiments, the conjugate compound is administered intranasally. In some instances, the conjugate compound is administered intraocularly (e.g., topically such as on the surface or just below the surface of the eye or intravitreally) to the subject. In other instances, the conjugate compound is administered intracisternally to the subject. In yet other instances, the conjugate compound is administered intrathecally to the subject. In still other instances, the conjugate compound is administered intravitreally to the subject. In yet other instances, the conjugate compound is administered topically or transdermally to the subject. In other instances, the conjugate compound is administered to the subject by injection, such as by subcutaneous injection, intramuscular injection, intravitreal injection, intraocular injection, intracisternal injection or intrathecal injection. And in still other instances, the conjugate compound may be administered and exert a “controlled release” effect in nerve tissue due to either the pharmacologic properties of the drug, whether via direct delivery to the nerve cell body or by signalling effects, and/or the biologic properties of the neurotrophin, also via direct delivery via one or two possible transport mechanisms and/or via the ‘steady state’.

In certain embodiments, protocols may include multiple dosage intervals. By “multiple dosage intervals,” it is meant that two or more dosages of the conjugate compound composition are administered to the subject in a sequential manner. In practicing methods of the present disclosure, treatment regimens may include two or more dosage intervals, such as three or more dosage intervals, such as four or more dosage intervals, such as five or more dosage intervals, including ten or more dosage intervals.

The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer.

In certain embodiments, compositions of the invention can be administered prior to, concurrent with, or subsequent to other therapeutic agents for treating the same or an unrelated condition. If provided at the same time as another therapeutic agent, compositions having the subject conjugate compounds may be administered in the same or in a different composition. By “concurrent therapy,” the intended administration of which to a subject is such that the therapeutic effect of the combination of the substances is caused in the subject undergoing therapy. For example, concurrent therapy may be achieved by administering an anti-inflammatory agent conjugate compound as described above with a pharmaceutical composition having at least one other agent, such as a second anti-inflammatory agent, immunosuppressant, steroid, analgesic, anesthetic, antihypertensive, chemotherapeutic, among other types of therapeutics, which in combination make up a therapeutically effective dose, according to a particular dosing regimen. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

Although the dosage used in treating a subject will vary depending on the clinical goals to be achieved, a suitable dosage range of the conjugate compound is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility and/or sensitivity of the subject to the intended beneficial pharmacologic effects and/or to the intended or unintended side effects.

In some embodiments, a suitable dose of the active agent conjugate is in the range of from about 1 mg/kg body weight to about 500 mg/kg body weight, e.g., from about 5 mg/kg body weight to about 500 mg/kg body weight, from about 10 mg/kg body weight to about 500 mg/kg body weight, from about 20 mg/kg body weight to about 500 mg/kg body weight, from about 30 mg/kg body weight to about 500 mg/kg body weight, from about 40 mg/kg body weight to about 500 mg/kg body weight, from about 50 mg/kg body weight to about 500 mg/kg body weight, from about 60 mg/kg body weight to about 500 mg/kg body weight, from about 70 mg/kg body weight to about 500 mg/kg body weight, from about 80 mg/kg body weight to about 500 mg/kg body weight, from about 90 mg/kg body weight to about 500 mg/kg body weight, from about 100 mg/kg body weight to about 500 mg/kg body weight, from about 200 mg/kg body weight to about 500 mg/kg body weight, from about 300 mg/kg body weight to about 500 mg/kg body weight, or from about 400 mg/kg body weight to about 500 mg/kg body weight.

In some embodiments, a suitable dose of the active agent conjugate, is in the range of from about 1 mg/kg body weight to about 5 mg/kg body weight, from about 5 mg/kg body weight to about 10 mg/kg body weight, from about 10 mg/kg body weight to about 20 mg/kg body weight, from about 20 mg/kg body weight to about 30 mg/kg body weight, from about 30 mg/kg body weight to about 40 mg/kg body weight, from about 40 mg/kg body weight to about 50 mg/kg body weight, from about 50 mg/kg body weight to about 100 mg/kg body weight, or from about 100 mg/kg body weight to about 500 mg/kg body weight.

In some embodiments, a single dose of the active agent conjugate is administered. In other embodiments, multiple doses of the conjugate compound are administered. Where multiple doses are administered over a period of time, the conjugate compound is administered, e.g., twice daily (qid), daily (qd), every other day (qod), every third day, three times per week (tiw), or twice per week (biw) over a period of time. For example, the conjugate compound is administered qid, qd, qod, tiw, or biw over a period of from one day to about 2 years or more. For example, the conjugate compound is administered at any of the aforementioned frequencies for one week, two weeks, one month, two months, six months, one year, or two years, or more, depending on various factors.

### PHARMACEUTICAL COMPOSITIONS AND DOSE UNITS

As described above, methods include administering a conjugate compound to treat neuroinflammation (e.g., COVID-19 mediated encephalitis). In some embodiments, the subject conjugate compounds are administered as compositions that include a pharmaceutically acceptable carrier. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins: Pharmaceutical Dosage Forms and Drug Delivery Systems (1999) H. C. Ansel et al., eds 7th ed., Lippincott, Williams, & Wilkins: and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly (ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Conjugate compositions may be formulated into compositions suitable for delivery to a subject or for contacting with a nerve cell by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols, and/or formulated by lyophilizing.

In certain instances, compositions of interest are formulated for injection such as by subcutaneous injection, intramuscular injection, intravitreal injection, intracisternal injection or intrathecal injection. In other instances, compositions are formulated to be administered intranasally to the subject. In other instances, compositions are formulated to be administered intraocularly to the subject. In still other instances, compositions are formulated to be administered intracisternally to the subject. In yet other instances, compositions are formulated to be administered intrathecally to the subject. In still other instances compositions are formulated to be administered intravitreally to the subject. In yet other instances, compositions are formulated to be administered topically or transdermally to the subject.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride: and sugars e.g., mannitol, dextrose, sucrose, and the like. In some embodiments, the aqueous buffer further includes a non-ionic surfactant such as polysorbate 20 or 80. In some instances, compositions of interest further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch: with binders, such as crystalline cellulose, cellulose derivatives, acacia, corn starch or gelatins: with disintegrators, such as corn starch, potato starch or sodium carboxymethylcellulose: with lubricants, such as talc or magnesium stearate: and if desired, with diluents, buffering agents, moistening agents, preservatives and flavoring agents.

Where the composition is formulated for injection, the conjugate compounds may be formulated by dissolving, suspending or emulsifying them in an aqueous or nonaqueous solvent, such as vegetable or other similar oils, synthetic aliphatic acid glycerides, esters of higher aliphatic acids or propylene glycol: and if desired, with conventional additives such as solubilizers, isotonic agents, suspending agents, emulsifying agents, stabilizers and preservatives.

Dose units of the present disclosure can be made using manufacturing methods available in the art and can be of a variety of forms suitable for injection (including intracisternal, intrathecal, intravenous, intramuscular, subcutaneous, intravitreal and intraocular) administration, for example as a solution, suspension, solution, lyophilate or emulsion. The dose unit can contain components conventional in pharmaceutical preparations, e.g. one or more carriers, binders, lubricants, excipients (e.g., to impart controlled release characteristics in addition to any controlled release effects which may be contributed by conjugating the composition to a neurotrophin), pH modifiers, coloring agents or further active agents.

Dose units provided as liquid dose units can have a total weight of from about 1 microgram to about 1 gram, and can be from about 5 micrograms to 1.5 grams, from about 50 micrograms to 1 gram, from about 100 micrograms to 1 gram, from 50 micrograms to 750 milligrams, and may be from about 1 microgram to 2 grams.

Dose units can comprise components in any relative amounts. For example, dose units can be from about 0.1% to 99% by weight of active ingredients (i.e., conjugate compound) per total weight of dose unit. In some embodiments, dose units can be from 10% to 50%, from 20% to 40%, or about 30% by weight of active ingredients per total weight dose unit.

Dose units can be provided in a variety of different forms and optionally provided in a manner suitable for storage. For example, dose units can be disposed within a container suitable for containing a pharmaceutical composition. The container can be, for example, a bottle (e.g., with a closure device, such as a cap, a vial, an ampule (for single dose units), a dropper, thin film, a tube and the like.

Containers can include a cap (e.g., screw cap) that is removably connected to the container over an opening through which the dose units disposed within the container can be accessed.

Containers can include a seal which can serve as a tamper-evident and/or tamper-resistant element, which seal is disrupted upon access to a dose unit disposed within the container. Such seal elements can be, for example, a frangible element that is broken or otherwise modified upon access to a dose unit disposed within the container. Examples of such frangible seal elements include a seal positioned over a container opening such that access to a dose unit within the container requires disruption of the seal (e.g., by peeling and/or piercing the seal). Examples of frangible seal elements include a frangible ring disposed around a container opening and in connection with a cap such that the ring is broken upon opening of the cap to access the dose units in the container. In certain embodiments, the composition is a lyophilized composition and the subject conjugate compounds in the composition are reconsistituted at the point of care. In certain instances, the container is a reconstistute at the point of care pen-injectable.

Liquid dose units can be placed in a container (e.g., bottle or ampule) of a size and configuration adapted to maintain stability of dose units over a period during which the dose units are dispensed into a prescription. For example, containers can be sized and configured to contain 10, 20, 30, 40, 50, 60, 70, 80, 90, 100 or more single liquid dose units. The containers can be sealed or resealable. The containers can packaged in a carton (e.g., for shipment from a manufacturer to a pharmacy or other dispensary). Such cartons can be boxes, tubes, or of other configuration, and may be made of any material (e.g., cardboard, plastic, and the like). The packaging system and/or containers disposed therein can have one or more affixed labels (e.g., to provide information such as lot number, dose unit type, manufacturer, and the like).

The container can include a moisture barrier and/or light barrier, e.g., to facilitate maintenance of stability of the active ingredients in the dose units contained therein. The container can be adapted to contain a single dose unit or multiples of a dose unit. The container can include a dispensing control mechanism, such as a lock out mechanism that facilitates maintenance of dosing regimen.

Dose units can be provided in a container in which they are disposed, and may be provided as part of a packaging system (optionally with instructions for use). For example, dose units containing different amounts of the conjugate compounds can be provided in separate containers, which containers can be disposed with in a larger container (e.g., to facilitate protection of dose units for shipment). For example, one or more dose units as described herein can be provided in separate containers, where dose units of different compositions are provided in separate containers, and the separate containers disposed within package for dispensing.

In some embodiments, the composition is a sustained release composition that is configured to deliver the active agent conjugate to the subject over a predetermined period of time, such as for 1 minute or more, such as for 5 minutes or more, such as for 10 minutes or more, such as for 15 minutes or more, such as for 30 minutes or more, such as for 60 minutes or more, such as for 2 hours or more, such as from 4 hours or more, such as for 6 hours or more, such as for 12 hours or more, such as for 24 hours or more and including for 168 hours or more. In certain embodiments, the composition is formulated to deliver the active agent conjugate to the subject over 1 month or more.

In other embodiments, the composition is formulated as an immediate release composition is formulated to release 50% or more of the active agent conjugate or a pharmaceutically acceptable salt thereof within 10 minutes or less of administration of the composition to the subject, such as 60% or more, such as 75% or more, such as 90% or more, such as 95% or more and including 99% or more within 10 minutes or less of administration of the composition to the subject. In certain instances, the composition is formulated to release 50% or more of the active agent conjugate or a pharmaceutically acceptable salt thereof immediately after contacting the composition to the eye of the subject, such as 60% or more, such as 75% or more, such as 90% or more, such as 95% or more and including 99% or more immediately after contacting the composition to the subject.

In some embodiments, the subject compositions are formulated for delayed onset immediate release, an effect which may be due to one or more of the combination of several factors to include but not limited to formulation: to the direct delivery or indirect e.g. signalling properties of the small molecule therapeutic agent; and/or to the pharmacokinetics associated with the rate of absorption of the neurotrophin-Trk component of the composition. By delayed onset immediate release is meant that the composition is formulated to delay release of the active agent conjugates for a predetermined period of time after which, the active agent conjugate is immediately released. In some instances, the predetermined period of delay time may be 5 minutes or longer, such as 10 minutes or longer, such as 15 minutes or longer, such as 20 minutes or longer and including 30 minutes or longer. In some embodiments, the delayed onset immediate release composition is formulated such that 20% or less of the active agent conjugate in the composition is released approximately 20 minutes after contacting the eye of the subject and 75% or more of the active agent conjugate in the composition is released approximately 30 minutes thereafter. In these embodiments, 20% or less of the active agent conjugate in the composition is released approximately 20 minutes after administration to the subject, such as 15% or less, such as 10% or less, such as 5% or less, such as 3% or less and including 1% or less of the active agent conjugate is released approximately 20 minutes after administration to the subject. After the predetermined delay period (e.g., 20 minutes), the delayed onset immediate release active agent conjugate is formulated to release 75% or more of the active agent conjugate approximately 30 minutes thereafter, such as 80% or more, such as 85% or more, such as 90% or more, such as 95% or more, such as 97% or more and including 99% or more. In certain embodiments, after the predetermined delay period after administration, the composition is formulated to release 100% of the active agent conjugate.

Aspects of the invention further include kits, where kits include an active agent conjugate having an anti-inflammatory agent covalently bonded to rhBDNF or a subunit thereof and an applicator configured for intranasal, intracisternal or intravitreal administration of the active agent conjugate to a subject diagnosed as having neuroinflammation. In some instances, the applicator includes a syringe, needle or other component suitable for administering a fluidic or solid (e.g., powdered solid, lyophilized) composition to a subject intranasally, intracisternally or intravitreally. Kits may further include components for reconstituting an active agent conjugate composition, such as a buffer (e.g., PBS buffer, normal saline solution) as well as containers (e.g., syringe) for mixing components of the composition.

In some embodiments, kits include 2 or more of the components of the subject compositions disclosed herein, such as 3 or more and including 5 or more. In some instances, the kits can include one or more assay components (e.g., labeled reagents, buffers, etc.). In some instances, the kits may further include a patient sample collection device, e.g., a lance or needle configured to prick skin to obtain a whole blood sample, a pipette, etc., as desired such as for diagnosing a subject as having neuroinflammation.

The various assay components of the kits may be present in separate containers, or some or all of them may be pre-combined. For example, in some instances, one or more components of the kit, e.g., active agent conjugate compound, buffer, syringe, needle, nasal applicator are present in a sealed pouch, e.g., a sterile foil pouch or envelope.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

Aspects, including embodiments, of the subject matter described herein may be beneficial alone or in combination, with one or more other aspects or embodiments. Without limiting the description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for treating neuroinflammation, the method comprising administering to a subject in need thereof an active agent conjugate comprising one or more active agent compounds covalently bonded to a protein, peptide or peptidomimetic that binds selectively to a neurotrophin receptor.
2. The method according to 1, wherein the method further comprises diagnosing the subject as having neuroinflammation.
3. The method according to any one of claims 1-2, wherein the neuroinflammation comprises encephalitis.
4. The method according to 3, wherein the neuroinflammation comprises viral encephalitis.
5. The method according to 4, wherein the neuroinflammation comprises COVID-19 associated encephalitis.
6. The method according to any one of 1-2, wherein the neuroinflammation comprises inflammation of the optic nerve.
7. The method according to 6, wherein the neuroinflammation comprises acute disseminated encephalomyelitis.
8. The method according to 7, wherein the neuroinflammation comprises COVID-19 associated acute disseminated encephalomyelitis.
9. The method according to any one of 1-2, wherein the neuroinflammation comprises inflammation of vitreous retinal ganglion cells.
10. The method according to any one of 1-9, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.
11. The method according to 10, wherein the protein, peptide or peptidomimetic is a recombinant human neurotrophic factor.
12. The composition according to 10, wherein the protein, peptide or peptidomimetic comprises a B-subunit of NGF, rhNGF or rhBDNF.
13. The method according to any one of 10-12, wherein the protein, peptide or peptidomimetic is neurotrophic factor that binds tropomyosin kinase A (TrkA).
14. The method according to any one of 1-13, wherein the active agent is an anti-inflammatory agent.
15. The method according to 14, wherein the anti-inflammatory agent is a glucocorticoid.
16. The method according to 15, wherein the glucocorticoid is fluocinolone acetonide.
17. The method according to any one of 1-13, wherein the active agent is antimicrobial compound.
18. The method according to 17, wherein the antimicrobial compound is an antiviral compound.
19. The method according to any one of 1-13, wherein the active agent is a compound configured to block receptor binding of neuropilin-1 (NRP1).
20. The method according to 19, wherein the active agent is an antibody configured to block receptor binding of NRP1.
21. The method according to 19, wherein the active agent is a small molecule compound configured to block receptor binding of NRP1.
22. The method according to any one of 1-21, wherein the active agent conjugate is administered to the subject intranasally, intravitreally or intracisternally.
23. A kit comprising:
   an active agent conjugate comprising an anti-inflammatory agent covalently bonded to rhNGF or a subunit thereof; and
   an applicator configured for intranasal administration of the active agent conjugate to a subject diagnosed as having neuroinflammation.
24. The kit according to 23, wherein the anti-inflammatory agent is a glucocorticoid.
25. The kit according to 24, wherein the glucocorticoid is fluocinolone acetonide.
26. The kit according to any one of claims 23-25, wherein the subject is diagnosed as having encephalitis.
27. The kit according to 26, wherein the subject is diagnosed as having viral encephalitis.
28. The kit according to 27, wherein the subject is diagnosed as having COVID-19 associated encephalitis.
29. A kit comprising:
   an active agent conjugate comprising an anti-inflammatory agent covalently bonded to rhBDNF or a subunit thereof; and
   an applicator configured for intranasal, intracisternal or intravitreal administration of the active agent conjugate to a subject diagnosed as having neuroinflammation.
30. The kit according to 29, wherein the anti-inflammatory agent is a glucocorticoid.
31. The kit according to 30, wherein the glucocorticoid is fluocinolone acetonide.
32. The kit according to any one of 29-31, wherein the neuroinflammation comprises inflammation of the optic nerve.
33. The kit according to 32, wherein the subject is diagnosed as having acute disseminated encephalomyelitis.
34. The kit according to 33, wherein the subject is diagnosed as having COVID-19 associated acute disseminated encephalomyelitis.
35. A method for treating a neurological condition, the method comprising administering to a subject in need thereof an active agent conjugate comprising one or more active agent compounds covalently bonded to a protein, peptide or peptidomimetic that binds selectively to a neurotrophin receptor.
36. The method according to 35, wherein the neurological condition comprises a brain injury.
37. The method according to 36, wherein the brain injury comprises chronic traumatic encephalopathy.
38. The method according to 35, wherein the neurological condition comprises brain cancer.
39. The method according to 38, wherein the brain cancer comprises a brain tumor selected from the group consisting of an astrocytoma, glioblastoma multiforme, meningioma, ependymoma, oligodendroglioma, cancer of the pituitary gland, craniopharyngioma, pineal region tumor and medulloblastoma.
40. The method according to any one of 38-39, wherein the brain cancer is caused by exposure to one or more toxins.
41. The method according to 40, wherein the brain cancer is caused by exposure to a toxin released in burn pits.
42. The method according to any one of 40-41, wherein the one or more toxins is selected from the group consisting of acetaldehyde, acheson process, acid mists, aflatoxins, 4-aminobiphenyl, areca nut, aristolochic acid, arsenic and inorganic arsenic compounds, asbestos, auramine, azathioprine, benzene, benzidine and dyes metabolized to benzidine, benzo[a]pyrene, beryllium, betel quid, bis(chloromethyl)ether and chloromethyl methyl ether, busulfan, 1,3-Butadiene, cadmium, chlorambucil, chlornaphazine, chromium (VI), clonorchis sinensis, coal, coal gasification, coal-tar distillation, coal-tar pitch, coke production, cyclophosphamide, cyclosporine, 1,2-dichloropropane, diethylstilbestrol (DES), diesel engine exhaust, erionite, ethanol, ethylene oxide, etoposide, fluoro-edenite fibrous amphibole, formaldehyde, haematite, isopropyl alcohol manufacture using strong acids, leather dust, lindane, magenta, melphalan, methoxsalen (8-methoxypsoralen) plus ultraviolet A radiation (PUVA), methyl-CCNU, 4,4′-Methylenebis(chloroaniline) (MOCA), 2-naphthylamine, nickel, N′-Nitrosonornicotine (NNN) and 4-(N-Nitrosomethylamino)-1-(3-pyridyl)-1-butanone (NNK), 3,4,5,3′,4′-Pentachlorobiphenyl (PCB-126), 2,3,4,7,8-pentachlorodibenzofuran, pentachlorophenol, phenacetin, phosphorus-32, plutonium polychlorinated biphenyls (PCBs), dioxin, iodine-131, radium-224, radium-226, radium-228, radon-222, semustine (methyl-CCNU), 2,3,7,8-Tetrachlorodibenzo-para-dioxin (TCDD), ortho-Toluidine, treosulfan, trichloroethylene, vinyl chloride, welding fumes and wood dust.
43. The method according to 35, wherein the neurological condition comprises multiple sclerosis.
44. The method according to 43, wherein the neurological condition comprises relapse-remitting multiple sclerosis.
45. The method according to 42, wherein the neurological condition comprises progressive multiple sclerosis.
46. The method according to any one of 43-45, wherein the active agent comprises bexarotene or a pharmaceutically acceptable salt thereof.
47. The method according to 35, wherein the neurological condition comprises Alzheimer's disease.
48. The method according to 35, wherein the neurological condition comprises Parkinson's disease.
49. The method according to 35, wherein the neurological condition comprises neuroinflammation.
50. The method according to 49, wherein the neuroinflammation comprises chronic pain.
51. The method according to 50, wherein the chronic pain is associated with or caused by one or more of headache, migraine, trigeminal neuralgia and herpes keratitis.
52. The method according to any one of 49-51, wherein the neuroinflammation is associated with or caused by viral infection.
53. The method according to 52, wherein the viral infection comprises infection by one or more of Herpes Zoster Virus (VZV) infection, Herpes Simplex Virus (HSV-1 or HSV-2), SARS-COV-2 virus. Epstein-Barr virus, Hepatitis B virus, Hepatitis C virus, Human immunodeficiency virus type 1 (HIV-1), Human papilloma virus (HPV), Human T-cell lymphotropic virus type I (HTLV-1) and Kaposi sarcoma herpesvirus (KSHV).
54. The method according to 53, wherein the neuroinflammation is associated with or caused by SARS-COV-2 virus infection.
55. The method according to any one of 49-54, wherein the neuroinflamation comprises encephalitis.
56. The method according to 55, wherein the neuroinflammation comprises viral encephalitis.
57. The method according to 56, wherein the neuroinflammation comprises COVID-19 associated encephalitis.
58. The method according to any one of 35-57, wherein the protein, peptide or peptidomimetic is selected from the group consisting of nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof.
59. The method according to 58, wherein the protein, peptide or peptidomimetic is a recombinant human neurotrophic factor.
60. The composition according to 58, wherein the protein, peptide or peptidomimetic comprises a B-subunit of NGF, rhNGF or rhBDNF.
61. The method according to any one of 58-60, wherein the protein, peptide or peptidomimetic is neurotrophic factor that binds tropomyosin kinase A (TrkA).
62. The method according to any one of 35-61, wherein the active agent is an anti-inflammatory agent.
63. The method according to 62, wherein the anti-inflammatory agent is a glucocorticoid.
64. The method according to 63, wherein the glucocorticoid is fluocinolone acetonide.
65. The method according to any one of 35-61, wherein the active agent is antimicrobial compound.
66. The method according to 65, wherein the antimicrobial compound is an antiviral compound.
67. The method according to 66, wherein the antiviral compound comprises plitidepsin.
68. The method according to any one of 35-61, wherein the active agent is a compound configured to block receptor binding of neuropilin-1 (NRP1).
69. The method according to 68, wherein the active agent is an antibody configured to block receptor binding of NRP1.
70. The method according to 69, wherein the active agent is a small molecule compound configured to block receptor binding of NRP1.
71. The method according to any one of 35-61, wherein the active agent comprises an antibody comprising activity against one or more viruses.
72. The method according to 71, wherein the antibody comprises activity against SARS-CoV-2 coronavirus or a variant thereof.
73. The method according to 72, wherein the antibody comprises activity against to a SARS-COV-2 coronavirus variant having one or more mutations selected from K417N, L452R, E484K, N501K, N501Y, D614G and P681H.
74. The method according to 72, wherein the antibody comprises activity against one or more of a SARS-COV-2 coronavirus of lineage B.1.1.207, a SARS-COV-2 coronavirus of lineage B.1.1.7, a SARS-COV-2 coronavirus Cluster 5, a SARS-COV-2 coronavirus variant 501.V2, a SARS-COV-2 coronavirus of lineage P.1 and a SARS-COV-2 coronavirus of lineage B.1.429.
75. The method according to any one of 71-74, wherein the antibody is a monoclonal antibody.
76. The method according to any one of 72-75, wherein the antibody is selected from bamlanivimab (LY-COV555), casirivimab, imdevimab and etesevimab and combinations thereof.
77. The method according to any one of 35-76, wherein the active agent conjugate is administered to the subject intranasally, intravitreally or intracisternally.
78. The method according to 77, wherein the active agent conjugate is administered to the subject intranasally.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Mass Spectroscopy and Dye-Adduct-Ratio (DAR)

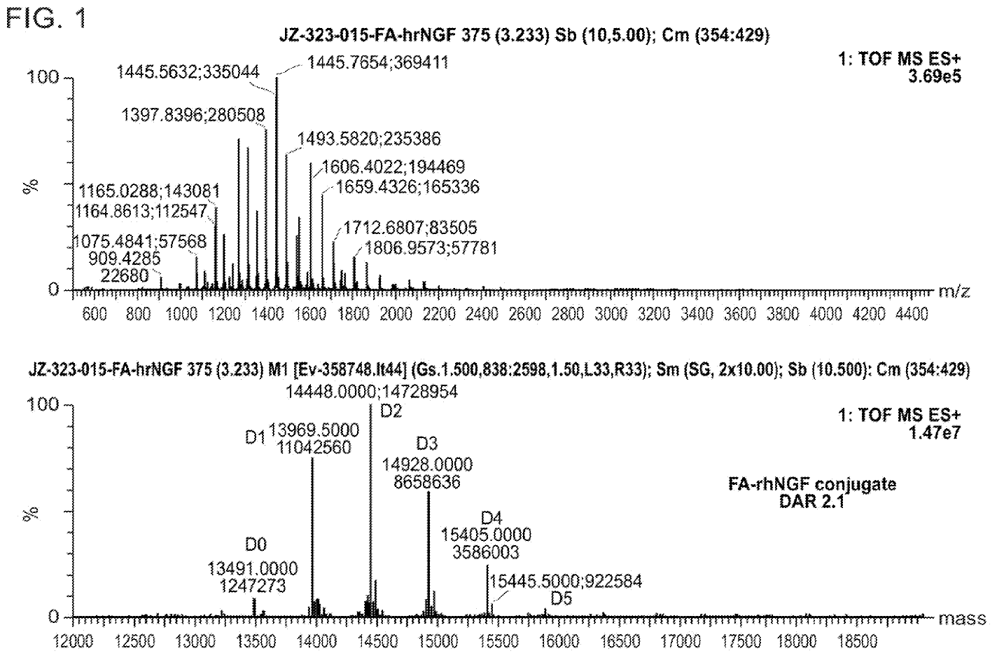

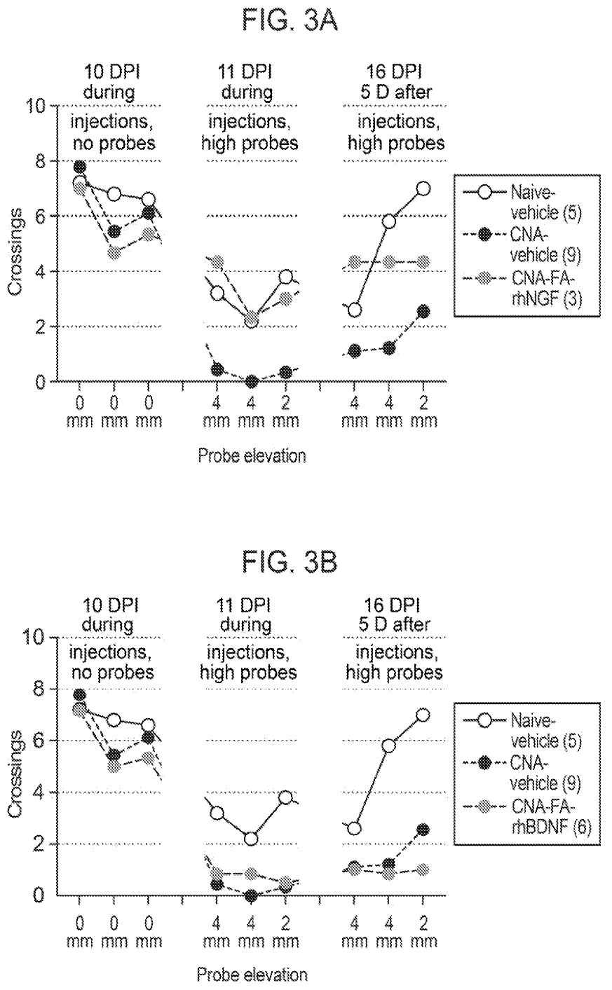

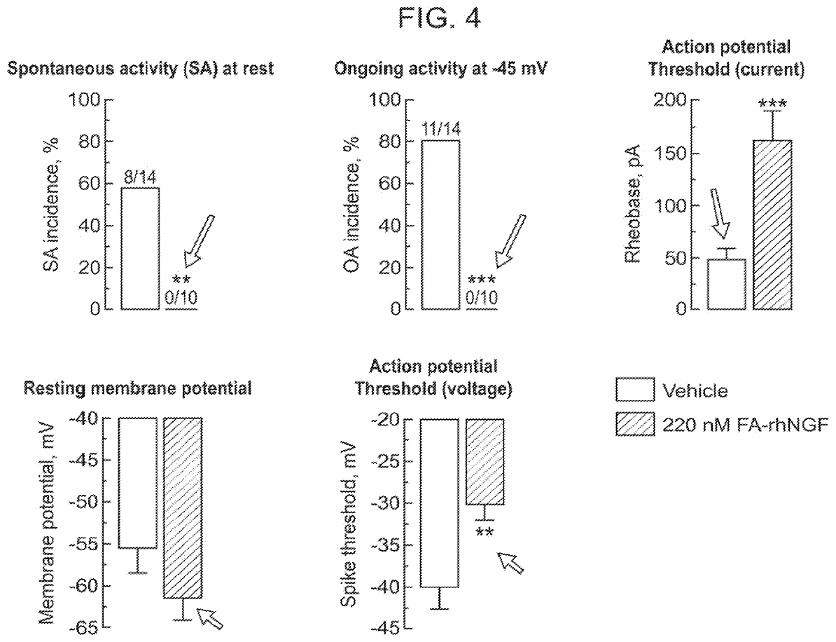

Standard analytics (mass spectroscopy, MS) and dye-adduct-ratio (DAR) were used to characterize and to guide synthesis. FIG. 1 depicts mass spectrogram (upper) and DAR 2.1 analytics (lower) from FA-rhNGF (DAR 1.95). Based on the analytics, the synthetic protocol for preparing the FA-rhNGF conjugate is reproducible and reliable analytical results were obtained. Anti-inflammatory activity of FA-rhNGF was assay in a rat model of pain (FIGS. 3 and 4).

### Bioassay of Neuronal Survival in Primary Neurons, in Superior Cervical Ganglia

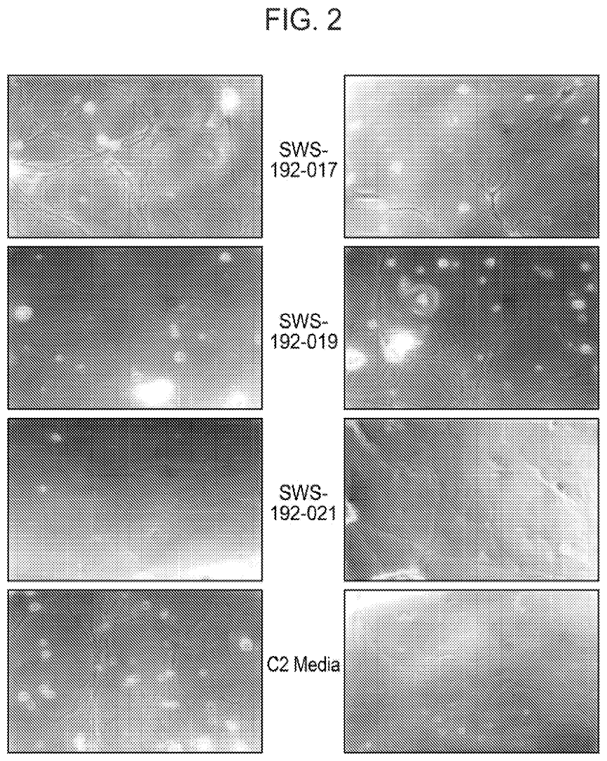

Superior cervical ganglia (SCG) neurons in primary cultures were used to study sympathetic neurons. The SCG assay was adapted to compartmented cultures and updated to grow primary neurons in microfluidic devices SCG neurons were harvested from E-21 Sprague Dawley rat pups. If labeled NGF has similar bioavailability to the unlabeled NGF, i.e., TrkA binding sites remain intact. FIG. 2 (right) compares left panels of study article with right panels showing treatment with NGF only. Since both images appear to show neuronal survival, it is concluded that TrkA binding is intact.

### In Vivo Activity

FIG. 3A depicts the pharmacologic activity of FA-rhNGF in a rat model of inflammatory pain. The contiguous neuritis and axotomy (CNA) chronic pain model combines aspects of peripheral nerve transection (spared nerve injury, SNI) with chronic constriction injury (CCI). The CCI model was adapted to distinguish components of neuro-inflammatory and axon injury in neuropathic pain. Neuro-inflammation of the tibial nerve and its sensory axons is produced by very loose, non-constricting looping of the tibial nerve with 4 inflammogenic chromic gut sutures: axotomy of nearby sensory axons is produced by transection of the sural nerve. In the CNA model, after injection concurrently mid-plantar and into gastrocnemius muscle, FA-rhNGF can be transported axonally to L4 DRG and to L5 DRG. In FIG. 3A, FA-rhNGF revealed statistically significant evidence for amelioration of mechanical hyperalgesia, as assessed by the MC test to measure how often a rat voluntarily crosses noxious probes in exploring a novel environment. The control FA-rhBDNF is given in FIG. 3B. The control groups were combined to increase the N's and permit statistical tests within each study. At 11 DPI, the total number of crossings per rat in the CNA-FA-rhNGF group was significantly greater than the crossings in the CNA-vehicle group (*, p<0.05).

On Day 28 suppression injected on Days 0-3 in vitro was recorded after overnight incubation of spinal cord injury-induced hyperactive nociceptors showing evidence of the pharmacologic and durability of the activity of FA as drug payload in FA-rhNGF, in electrophysiology studies. Pre-treatment with FA-rhNGF significantly reduced spontaneous activity at resting membrane potential and ongoing activity when membrane potential was depolarized to-45 mV, and also reversed other properties, including voltage threshold. In vitro spontaneous activity (SA) at resting membrane potential and ongoing activity (OA) tested at a holding potential of −45 mV were both completely suppressed by overnight application of 220 nM FA-rhNGF. After SNA, less suppression was found in L5 DRG neurons (spinal nerve transected) than in intact L4 DRG. The strong suppression of SA and OA after SCI and in the L4 DRG neurons after SNA suggests that FA-rhNGF is especially effective in reducing hyperactivity triggered by inflammatory signals rather than direct cellular injury. This is consistent with nociceptor hyperactivity induced by inflammation being more important in driving chronic neuropathic pain than that induced by nociceptor axotomy and that FA-rhNGF suppresses pain-related hyperactivity in nociceptors induced by inflammation.

FIG. 4 depicts the suppression of in vitro SCI-induced hyperactivity of nociceptors by overnight incubation with FA-rhNGF. FA-rhNGF pre-treatment significantly reduced spontaneous activity at resting membrane potential and ongoing activity when membrane potential was depolarized to −45 mV. It also reversed other hyperexcitable properties, including the depression of action potential current threshold (rheobase) and voltage threshold, and to hyperpolarize resting membrane potential. In the SNA model, no effect of FA-rhNGF was found on hyperexcitability of previously axotomized L5 neurons (n=8) but it suppressed hyperexcitability properties of L4 neurons, which remained intact in vivo (n=7).

Cytokine analyses of DRG L3-L6 was conducted on Day 28 after injecting FA-rhNGF (6 μg 4X QD, ipsilateral) in the SNL model on Days 0-3. Results showed that FA-rhNGF acted in treated DRG on interferon-gamma, a biomarker for lumbar root irritation; ciliary neurotrophic factor: CD54, and CXCL7. The above shows that FA-rhNGF is pharmacologically active in vivo (FIG. 3) in the treatment of neuroinflammation; and FA-rhNGF has a durable effect (FIG. 4).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim: if such exact phrase is not used in a limitation in the claim, then 35 U.S.C. § 112 (f) or 35 U.S.C. § 112(6) is not invoked.

What is claimed is:

1. A method for treating neuroinflammation, the method comprising administering to a subject in need thereof an active agent conjugate comprising one or more active agent compounds covalently bonded to a protein, peptide or peptidomimetic that binds selectively to a neurotrophin receptor.

2. The method according to claim 1, wherein the neuroinflammation comprises viral encephalitis.

3. The method according to claim 1, wherein the neuroinflammation comprises inflammation of the optic nerve.

4. The method according to claim 1, wherein the neuroinflammation comprises acute disseminated encephalomyelitis.

5. The method according to claim 4, wherein the neuroinflammation comprises COVID-19 associated acute disseminated encephalomyelitis.

6. The method according to any one of claims 1-2, wherein the neuroinflammation comprises inflammation of vitreous retinal ganglion cells.

7. The method according to any one of claims 1-6, wherein the protein, peptide or peptidomimetic is selected from the group consisting of: nerve growth factor (NGF), brain-derived neurotrophic factor (BDNF), neurotrophic factor 3 (NT-3), neurotrophic factor 4/5 (NT-4/5), neurotrophic factor 6 (NT-6), neurotrophic factor 7 (NT-7), glial cell-derived neurotrophic factor (GDNF), ciliary neurotrophic factor (CNTF) and derivatives or fragments thereof; and
the protein, peptide or peptidomimetic comprises a β-subunit of NGF, rhNGF or rhBDNF.

8. The method according to any one of claims 1-7, wherein the active agent is a compound configured to block receptor binding of neuropilin-1 (NRP1), wherein:
the active agent is an antibody configured to block receptor binding of NRP1; or the active agent is a small molecule compound configured to block receptor binding of NRP1.

9. The method according to any one of claims 1-8, wherein the active agent conjugate is administered to the subject intranasally, intravitreally or intracisternally.

10. A method for treating a neurological condition, the method comprising administering to a subject in need thereof an active agent conjugate comprising one or more active agent compounds covalently bonded to a protein, peptide or peptidomimetic that binds selectively to a neurotrophin receptor.

11. The method according to claim 10, wherein the neurological condition is selected from the group consisting of a brain injury, chronic traumatic encephalopathy, Alzheimer's disease, Parkinson's disease, multiple sclerosis or a brain cancer selected from an astrocytoma, glioblastoma multiforme, meningioma, ependymoma, oligodendroglioma, cancer of the pituitary gland, craniopharyngioma, pineal region tumor and medulloblastoma.

12. The method according to any one of claims 10-11, wherein the active agent comprises bexarotene or a pharmaceutically acceptable salt thereof.

13. The method according to claim 10, wherein the neurological condition is neuroinflammation associated with or caused by viral infection.

14. The method according to claim 13, wherein the viral infection comprises infection by one or more of Herpes Zoster Virus (VZV) infection, Herpes Simplex Virus (HSV-1 or HSV-2), SARS-COV-2 virus. Epstein-Barr virus, Hepatitis B virus, Hepatitis C virus, Human immunodeficiency virus type 1 (HIV-1), Human papilloma virus (HPV), Human T-cell lymphotropic virus type I (HTLV-1) and Kaposi sarcoma herpesvirus (KSHV).

15. The method according to claim 14, wherein the neuroinflammation is associated with or caused by SARS-COV-2 virus infection.
